(19) United States
(12) Patent Application Publication
(10) Pub. No.: US 2012/0184810 A1
(43) Pub. Date: Jul. 19, 2012

(54) SELF-LOCKING CANNULA

(76) Inventor: Jean-Christophe ZECH, Lyon (FR)

(21) Appl. No.: 13/344,829

(22) Filed: Jan. 6, 2012

(30) Foreign Application Priority Data
Jan. 18, 2011 (FR) 11 00141

(51) Int. Cl. A61B 1/06; A61F 9/007; A61B 1/018

(52) U.S. Cl. 600/106

(57) ABSTRACT

Cannula intended to traverse the conjunctiva and/or the sclera of the eye in order to enable the passage of at least one tool intended to have an action in the intraocular volume, such as a suction instrument, an instrument for cutting—for example at high speed—the vitreous gel contained in the intraocular volume, a light-guide intended to transmit light in order to see what is happening in the intraocular volume or similar, the cannula including a tubular part defining a passage channel for the tool, which extends between a proximal opening intended to be outside the intraocular volume and a distal opening intended to be inside the intraocular volume.

### TECHNICAL FIELD

The present invention relates to an assembly composed of at least one cannula intended to traverse the conjunctiva and/or the sclera of the eye in order to enable the passage of a tool intended to have an action in the intraocular volume, such as a suction instrument, an infusion line, an instrument for cutting—for example at high speed—the vitreous gel contained in the intraocular volume, a light-guide intended to transmit light in order to see what is happening in the intraocular volume or similar, and a tool of the aforementioned type having appropriate dimensions in order to be able to pass through the cannula.

### BACKGROUND ART

The assemblies of this type of the prior art are not easy for the surgeon to use. In particular, the movement of the surgeon for displacing the tools often lacks precision, and, by virtue of the manipulations of the eye, for example when the surgeon causes an infusion line to pass through the cannula, it may happen that the assembly constituted by cannula and line comes out again towards the outside and physiological liquid comes to be injected underneath the retina, which is not desirable and may cause a detachment of the retina.

### DISCLOSURE OF THE INVENTION

The present invention aims to overcome the drawbacks of the prior art by proposing an assembly of the type mentioned above that gives the surgeon a greater sense of safety when he/she is manoeuvring his/her tool in the intraocular cavity, having the effect of improving safety for the patient and of accelerating the operations being carried out.

According to the invention, a cannula is as defined in claim 1, further developments being defined in the dependent claims 2 to 6.

By thus providing for locking the cannula in position when it is positioned through the sclera and/or the conjunctiva in order to form a passage through these walls for the tool, it is ensured that the cannula remains stably in position, which combats an untimely exit of the cannula and the tool that goes through it, notably a perfusion line, during the manipulation of the tool or of the eye, enabling the harmful consequences to be avoided that are linked with such an untimely exit, for example an injection of liquid underneath the retina in the case where the associated tool is a perfusion line. The cannula is thus self-locking.

According to a preferred and, notably, particularly simple embodiment of the invention, locking means are provided in the form of an annular groove formed on the outside of the cannula.

In particular, according to a preferred embodiment of the invention the groove is formed by a narrowing of the external casing of the cannula.

According to another preferred embodiment of the invention, the groove is formed by two annular stops projecting laterally from the cannula and spaced from one another so as to form the annular groove therebetween.

According to a preferred embodiment of the invention, the distal end of the cannula—that is to say, the end through which the cannula penetrates into the intraocular space—has a bevelled shape.

This bevel promotes a progressive and non-traumatic positioning of the cannula through the sclera.

According to another possible embodiment, also enabling a progressive positioning to be promoted, cannula of conical shape is provided over its length, for example from the stop as far as its distal end.

The present invention also relates to an assembly composed of at least one cannula according to the invention and at least one tool intended to have an action in the intraocular volume, such as a suction instrument, an instrument for cutting—for example at high speed—the vitreous gel contained in the intraocular volume, a light-guide intended to transmit light in order to see what is happening in the intraocular volume or similar, and having appropriate dimensions in order to be able to pass into the channel of the cannula, and also to an assembly composed of at least one cannula according to the invention and at least one tool intended to have an action in the intraocular volume, such as a suction instrument, an instrument for cutting—for example at high speed—the vitreous gel contained in the intraocular volume, a light-guide intended to transmit light in order to see what is happening in the intraocular volume or similar, and passing into the channel of the cannula.

### BRIEF DESCRIPTION OF THE DRAWINGS

By way of example, preferred embodiments of the invention will now be described, referring to the drawings in which:

FIG. 1 is a schematic sectional view of an eye into which two tools have been introduced by means of two cannulae according to the invention;

FIG. 2 is an enlarged view of a part of FIG. 1;

FIG. 3 is a perspective view of the cannula shown in FIGS. 1 and 2;

FIG. 4 is a perspective view of another embodiment of a cannula according to the invention; and

FIG. 5 is a perspective view of yet another embodiment of a cannula according to the invention.

### PREFERRED EMBODIMENTS FOR CARRYING OUT THE INVENTION

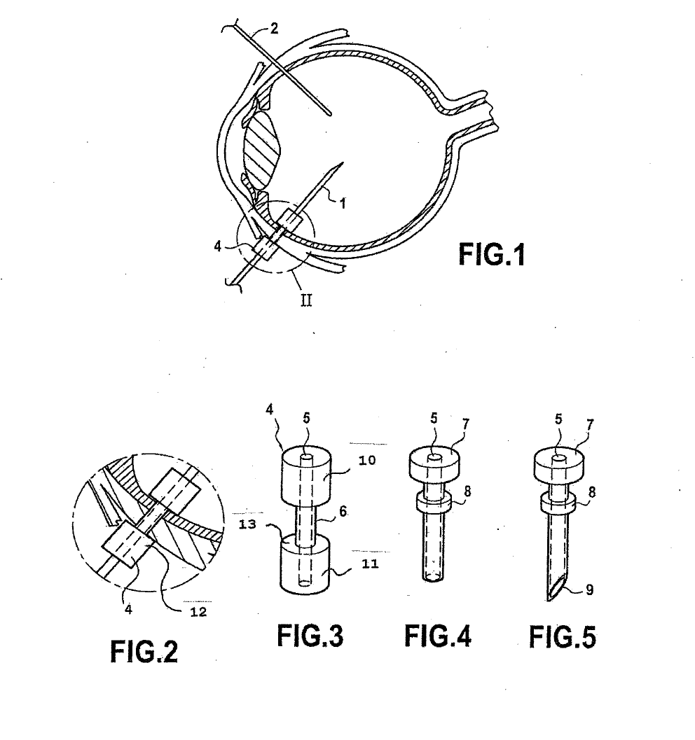

In FIG. 1 a sectional view of an eye is represented schematically. Tools 1 and 2, respectively a suction tool 1 and a tool 2 forming a light-guide for enabling the surgeon to see what he/she is doing with the tool 1, are inserted into the intraocular space of the eye. Each tool 1 and 2 passes into the eye by passing into a respective cannula 4 which ensures the crossing of the walls delimiting the intraocular cavity, namely the sclera and/or the conjunctiva.

The cannula 4 is constituted by a tubular part defining a passage which extends between a proximal opening 5 intended to be outside the intraocular volume and a distal opening opposite the opening 5 and intended to be located in the intraocular volume.

The cannula 4 is locked in a position in which it traverses the two walls and remains locked there by locking means which are constituted here by a hollowed-out annular groove 6 in the external surface or casing of the cannula. The cannula shown in FIGS. 1 and 2 is represented in more detail in FIG. 3. Said cannula is constituted by a tubular element having a larger outside diameter and defines in its interior a passage channel having an inside diameter. The groove 6 is hollowed out on the external surface of the cannula, substantially in its middle. The groove 6 thus forms a narrowing, the outside diameter of which is smaller than the largest outside diameter of the channel and slightly larger than the inside diameter of the channel. The extension in length—that is to say, between the proximal and distal openings 5 along the longitudinal direction of the channel of the cannula—of the groove 6 corresponds substantially to the thickness of the two walls formed by the sclera and the conjunctiva, so that once the cannula has been inserted through these two walls these two walls are sandwiched between the two shoulders—the upper shoulder 10 and the lower shoulder 11—delimiting the groove 6 and thus lock the cannula 4 in position. The contact between the plane distal face 12 of the upper or proximal shoulder 10 and the wall of the sclera and/or of the conjunctiva is effected along a plane, at least in part. The contact between the proximal plane face 13 of the distal or lower shoulder 11 and the wall delimiting the intraocular space (the sclera and/or the conjunctiva) is effected along a plane, at least in part.

The distance between the two distal and proximal plane faces 12 and 13 of the proximal and distal shoulders 10 and 11, respectively, is appreciably equal to or slightly smaller than the thickness of the wall or walls delimiting the intraocular space (the sclera and/or the conjunctiva).

Likewise, in the embodiments shown in FIG. 4 or 5 the lower or distal shoulder 8 has a proximal face equivalent to the face 13 of the shoulder 11 of the embodiment shown in FIGS. 1 to 3, and the shoulder 7 has a distal face equivalent to the face 12 of the shoulder 11, and the two shoulders 7 and 8 sandwich the two walls delimiting the intraocular cavity along respective plane contacts, as in the embodiment shown in FIGS. 1 to 3.

According to another embodiment, represented in FIG. 4, the cannula, still constituted by a tubular element forming a channel in its interior, includes a head 7 of larger diameter than the diameter of the tubular part of the cannula. On the other side, an annular stop 8 projects outside the cannula. Thus between the head 7 and the annular stop 8 an annular groove is formed which has the same function as the groove 6 in the embodiment shown in FIG. 3. In particular, for this groove the same thickness may be provided as that provided for the groove 6 of the embodiment shown in FIG. 3.

In the Figure an outside diameter of the stop has been represented which is smaller than that of the head. However, while remaining within the scope of the invention, identical diameters or, conversely, a larger diameter may be provided for the stop. However, the fact of providing a smaller diameter for the stop presents the additional advantage of facilitating the positioning of the cannula.

Represented in FIG. 5 is yet another embodiment corresponding appreciably to that shown in FIG. 4 but in which, in addition, the distal opening 9 of the cannula has a bevelled shape, ensuring a progressive and non-traumatic insertion into the incision of the walls of the sclera and of the conjunctiva when the cannula is inserted for the first time in order to be disposed across these walls.

This incision may be realised by mounting the cannula onto a knife which incises the scleral+conjunctival thickness, then by means of a clamp the surgeon holds the head of the cannula and withdraws the knife, the cannula being thus placed in position. This is the so-called one-step system. Other systems exist, for example a system that consists in positioning a gauge that makes it possible for the pars plana (the place where the incision has to be made) to be located, for an incision to be made with the knife, and then for the cannula to be inserted, the gauge helping to find the site of the incision. This system is known as the two-step system, by virtue of a tool which is introduced into the cannula and which is then withdrawn once the cannula has been installed.

The cannula is manufactured from a material that is standard in the field, in particular from metal such as stainless steel or from thermoplastic or plastic materials such as a polyimide.

What is claimed is:

1. Cannula intended to traverse the conjunctiva and/or the sclera of the eye in order to enable the passage of at least one tool intended to have an action in the intraocular volume, such as a suction instrument, an instrument for cutting—for example at high speed—the vitreous gel contained in the intraocular volume, a light-guide intended to transmit light in order to see what is happening in the intraocular volume or similar, the cannula including a tubular part defining a passage channel for the tool, which extends between a proximal opening intended to be outside the intraocular volume and a distal opening intended to be inside the intraocular volume, characterised in that means are provided for locking the cannula in position in a position in which it traverses the sclera and/or the conjunctiva, the locking opposing the displacement of the cannula towards the outside and towards the inside of the intraocular volume, the locking means having the form of an annular groove formed on the outside of the cannula so that, once the cannula has been inserted through the two walls of the conjunctiva and/or of the sclera, these latter are sandwiched between two shoulders (10, 11; 7, 8).

2. Cannula according to claim 1, characterised in that the faces (12, 13) of the shoulders (10, 11) intended to come into contact with the walls of the conjunctiva and/or of the sclera are plane.

3. Cannula according to claim 1, characterised in that the groove is formed by a narrowing of the external casing of the cannula.

4. Cannula according to claim 1, characterised in that the groove is formed by two annular stops projecting laterally from the cannula and spaced from one another so as to form the annular groove therebetween.

5. Cannula according to claim 1, characterised in that the distal end of the cannula—that is to say, the end through which the cannula penetrates into the intraocular space—is of bevelled shape.

6. Cannula according to claim 5, characterised in that the cannula has a conical shape, notably from the stop (8) as far as its distal end.

7. Assembly constituted by at least one cannula according to claim 1 and by at least one tool intended to have an action in the intraocular volume, such as a suction instrument, an instrument for cutting—for example at high speed—the vitreous gel contained in the intraocular volume, a light-guide intended to transmit light in order to see what is happening in the intraocular volume or similar, and having appropriate dimensions in order to be able to pass into the channel of the cannula.

8. Assembly constituted by at least one cannula according to claim 1 and by at least one tool intended to have an action in the intraocular volume, such as a suction instrument, an instrument for cutting—for example at high speed—the vitreous gel contained in the intraocular volume, a light-guide intended to transmit light in order to see what is happening in the intraocular volume or similar, and passing into the channel of the cannula.
